(19) United States
(12) Patent Application Publication
(10) Pub. No.: US 2018/0368717 A1
(43) Pub. Date: Dec. 27, 2018

(54) METHOD AND SYSTEM FOR RECOVERING OPERATING DATA OF A DEVICE FOR MEASURING BRAIN WAVES

(71) Applicant: RYTHM, Paris (FR)

(72) Inventors: Quentin SOULET DE BRUGIERE, Pyla Sur Mer (FR); Hugo MERCIER, PARIS (FR); Olivier TRANZER, Paris (FR); Adrien CATANAS, Asnieres (FR)

(21) Appl. No.: 16/060,834

(22) PCT Filed: Dec. 9, 2016
(86) PCT No.: PCT/FR2016/053307

§ 371 Date: Jun. 8, 2018

(30) Foreign Application Priority Data
Dec. 9, 2015 (FR) 1562080

(51) Int. Cl. A61B 5/0476; A61B 5/00; H04B 11/00; H04W 4/80

(52) U.S. Cl. CPC A61B 5/0476; A61B 5/0006; A61B 5/6803; H04W 4/80; A61B 5/0022; H04B 11/00

(57) ABSTRACT

A method for retrieving operating data from a measuring device measuring brain waves includes: a measurement signal acquisition on the measuring device; a first test step determining whether a primary connection can be established between the measuring device and a data processing server; if so, a step of primary transfer of operating data from the measuring device to the server; otherwise, a second test step for determining whether a secondary connection can be established between the measuring device and a portable relay device; if a secondary connection can be established, a step of secondary transfer of operating data from the measuring device to the portable relay device; a third test step determining whether a tertiary connection can be established between the portable relay device and the server; if so, a tertiary transfer step of the operating data of the portable relay device to the server.

### FIELD OF THE INVENTION

The present invention relates to methods and systems for retrieving operating data from a device for measuring brain waves of an individual.

### BACKGROUND OF THE INVENTION

There are known devices for measuring a person's brain waves, especially during a person sleeping, working or leisure period.

Such measuring devices usually comprise a helmet or headband provided with electrodes for measuring an encephalogram, and for example an electromyogram. The measurement device is worn by the person over a period of time, for example a user's sleeping period for a sleep tracking device. Such devices may further act on the brain function of the user, for example by means of sensory stimulators, for example sound stimulation means.

Document WO 2015/17563 describes an example of such a device for measuring the brain waves of a person.

In order to process the data collected by such a measurement device, in particular the electroencephalogram signals, it is usually necessary to call on a processing server because the computing power and the required memory are important. In addition, a processing server allows to centralize the data collected by a plurality of measurement devices and to store and process the data resulting from an acquisitions series. It is thus for example possible to implement learning or statistical calculations algorithms.

Yet, even when it is not used, the device for measuring brain waves is often kept in the room where the acquisition usually takes place, for example laying during the day on a bedside table in the bedroom in the case of a sleep tracking device.

It is also noted that access to the Internet is not always available in the different rooms of a house. In particular, the wireless router of a Wi-Fi network is frequently found in a living room of the house which can be far from the bedroom. This can be chosen by the user for practical and economic reasons, so as to limit the number of Wi-Fi routers, or because the user wants to limit its exposure to electromagnetic radiations during sleep.

As a result, the measurement device can in practice have significant difficulties in communicating with the Internet and therefore with the processing server. The operating data retrieval from a device for measuring brain waves of a person onto a data processing server can thus be delicate and delayed, unless the user is regularly required to perform a manual data transfer operation, which is obviously burdensome and time-consuming for the user.

The present invention is intended in particular to improve this situation.

### SUMMARY OF THE INVENTION

For this purpose, the invention firstly relates to a method for retrieving operating data from a device for measuring the brain waves of a person onto a data processing server, specifically intended to be implemented by a system comprising a data processing server, a portable relay device and a measuring device for measuring brain waves of a person,

the method comprising at least:

a) a working step in which, during a working period, a measurement signal (S) representative of a physiological signal of the person (P) is acquired by means of the measuring device, and said measurement signal is stored in a memory of said measuring device,

b1) a first connection test step, implemented after said working period, during which it is determined whether a primary connection can be established between the measuring device and the data processing server,

c1) if a primary connection can be established, a step of primary transfer of operating data from the measuring device to the data processing server, by means of said primary connection, said operating data being determined from the measurement signal,

b2) if a primary connection can not be established, a second connection test step, during which it is determined whether a secondary connection can be established between the measuring device and the portable relay device,

c2) if a secondary connection can be established, a step of secondary transfer of operating data from the measuring device to the portable relay device, by means of said secondary connection, said operating data being determined from the measurement signal,

b3) if a secondary transfer step has been implemented, a third connection test step, during which it is determined whether a tertiary connection can be established between the portable relay device and the data processing server,

c3) if a tertiary connection can be established, a tertiary transfer step of the operating data from the portable relay device to the data processing server, by means of said tertiary connection.

In preferred embodiments of the invention, one and/or another of the following arrangements may also be used:

the portable relay device is a device which is transportable by a user, in particular a base, a mobile phone, a smartphone, a tablet or a laptop;

the primary connection, the secondary connection and the tertiary connection each comprise a wireless communication;

the primary connection is implemented by means of a local wireless network connected to a wide area network, in particular a corporate wireless network or a home wireless network connected to the Internet;

the secondary connection is a wireless connection between the brain waves measuring device and the portable relay device, including an ultrasonic connection or a radio frequency connection such as a Bluetooth connection or a near-field communication;

the tertiary connection is implemented at least in part by means of a wireless network such as a cellular network or a local wireless network connected to the Internet, in particular a wireless network connected to the Internet or a home wireless network connected to the Internet;

the portable relay device is moved between the secondary transfer step and the tertiary transfer step;

the second connection test step comprises a first test sub-step during which it is determined whether a radio frequency connection can be established between the brain waves measuring device and the portable relay device,

if a radio-frequency connection can be established, the secondary connection is a radio-frequency connection,

if a radio frequency connection can not be established, a second test sub-step in which it is determined whether an ultrasonic connection can be established between the brain waves measuring device and the portable relay device,

if an ultrasonic connection can be established, the secondary connection is an ultrasonic connection;

the operating data transmitted from the brain waves measuring device to the data processing server during the primary transfer step comprise raw measurement data including the measurement signal;

the operating data transmitted during the secondary transfer step and the tertiary transfer step comprise processed measurement data, preferably do not include the measurement signal (S), even more preferably said operating data present a size at least ten times smaller than a size of the raw measurement data which include the measurement signal (S);

the processed measurement data is determined by implementing a predefined pattern recognition algorithm for recognizing predefined patterns in the measurement signal, including slow wave patterns, sleep spindle patterns, patterns associated with the waking and/or with the movements of the person,

and said processed measurement data comprises indicators relating to said predefined patterns, including a predefined pattern start time, duration, frequency and/or amplitude, and/or a number or frequency of a pattern which is predefined during the working period;

during the working step, an acoustic signal (A) is transmitted, audible by the person, and synchronized with a predefined temporal brain wave pattern (M1) of the person,

and the operating data transmitted during the primary transfer step comprises at least one stimulation parameter selected from a list comprising an acoustic stimulation pattern start time, duration, amplitude, spectrum and/or reference,

preferably the operating data transmitted during the secondary transfer step and during the tertiary transfer step also comprise said at least one stimulation parameter.

The invention also relates to a system comprising a data processing server, a portable relay device and a device for measuring the brain waves of a person,

wherein the measuring device comprises

acquisition means capable, during a working period, of acquiring at least one measurement signal which is representative of a physiological signal of the person (P),

a memory capable of storing said measurement signal, and

communication means suitable for

determining whether a primary connection can be established between the measuring device and the data processing server,

transferring data from the measuring device to the data processing server by means of a primary connection,

determining whether a secondary connection can be established between the measuring device and the portable relay device, and

transferring data from the measuring device to the portable relay device by means of a secondary connection,

wherein the portable relay device comprises communication means adapted to

determining whether a tertiary connection can be established between the portable relay device and the data processing server,

transferring data from the portable relay device to the data processing server by means of a tertiary connection.

The invention also related to a device for measuring brain waves of a person which is specifically intended to be integrated into a system as described above, the device comprising

acquisition means capable, during a working period, of acquiring at least one measurement signal which is representative of a physiological signal of the person (P),

a memory capable of storing said measurement signal, and

communication means suitable for

determining whether a primary connection can be established between the brain wave measuring device and a data processing server of a system according to the invention. transferring data from the measuring device to the data processing server by means of a primary connection,

determining whether a secondary connection can be established between the brain wave measuring device and a portable relay device of a system according to the invention, and

transferring data from the measuring device to the portable relay device by means of a secondary connection,

According to one embodiment, the arrangement further comprises transmission means designed to emit an acoustic signal, audible by the person, and synchronized with a predefined temporal brain wave pattern of the person.

Thanks to these arrangements, among other things, the operating data retrieval from the measuring device for measuring brain waves of a person on a processing server is facilitated, is less restrictive for the user, does not require displacing the measuring device, or any particular action from the user, is more reliable and is not delayed.

### DESCRIPTION OF THE DRAWINGS

Other features and advantages of the invention will emerge from the following description of several embodiments, given as non-limiting examples, with respect to the attached drawings.

On the drawings:

FIG. 1 is a schematic view of a device for measuring the brain waves of a person according to an embodiment of the invention,

FIG. 2 is a synoptic diagram of a system according to an embodiment of the invention comprising a measuring device, a portable relay device and a data processing server,

FIG. 3 is a synoptic diagram of a primary connection and a primary transfer of operating data between the measurement device and the data processing server of the system of FIG. 2, during the implementation of a method according to an embodiment of the invention,

FIG. 4 is a synoptic diagram of a secondary connection and a secondary transfer of operating data between the measuring device and the portable relay device of the system of FIG. 2, during the implementation of a method according to an embodiment of the invention,

FIG. 5 is a synoptic diagram of a tertiary connection and a tertiary transfer of operating data between the portable relay device and the data processing server of the system of FIG. 2, during the implementation of a method according to an embodiment of the invention,

FIG. 6 is a flowchart illustrating an embodiment of a method for retrieving operating data from a device for measuring the brain waves of a person onto a data processing server according to an embodiment of the invention,

FIG. 7 illustrates a temporal shape of a slow brain wave, an acoustic signal and predefined temporal patterns according to an exemplary embodiment of the invention.

In the different figures, the same references are used to designate elements that are identical or similar.

### DETAILED DESCRIPTION

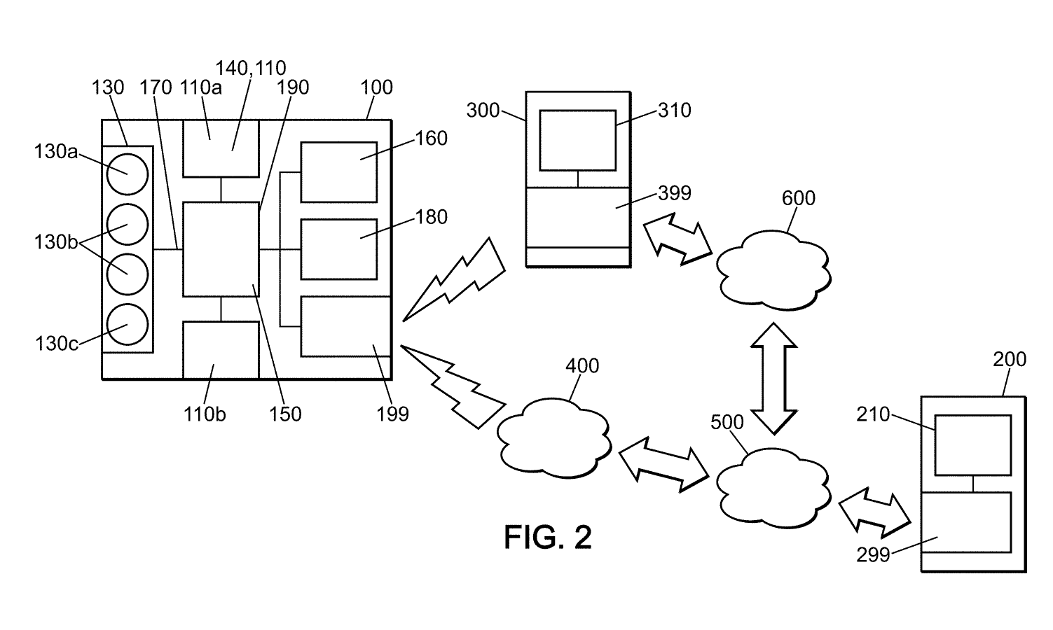

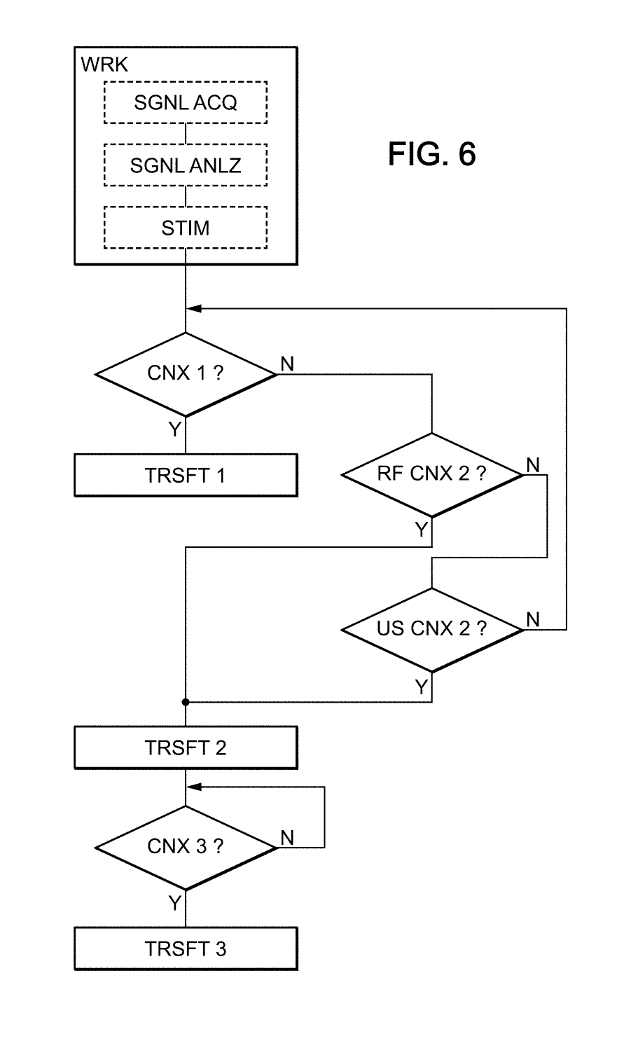

As illustrated in particular in FIGS. 2 and 6, the invention relates to a system 1 comprising a device 100 for measuring the brain waves of a person, a data processing server 200 and a portable relay device 300.

The system 1 is able to implement a method for retrieving operating data from the device for measuring the brain waves of a person P onto the data processing server which is in particular illustrated in FIG. 6.

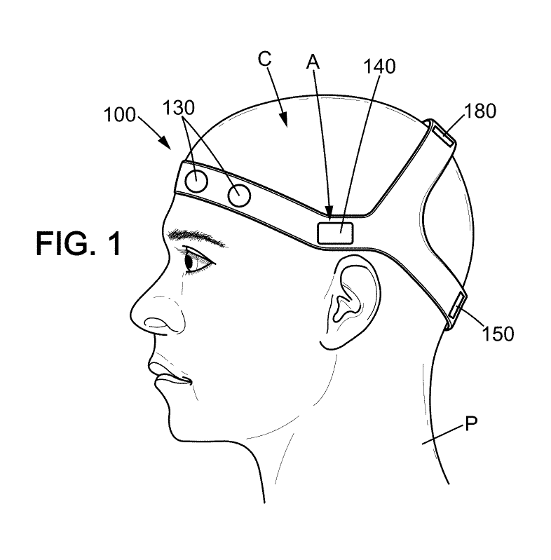

The device 100 is illustrated in FIGS. 1 and 2 and is for example adapted to be worn by the person P, for example on the head of the person P.

To this end, the device 100 may comprise one or more support elements 120 able to at least partially surround the head of the person P so as to be held there. The support elements 120 take for example the shape of one or more branches that can be arranged so as to surround the head of the person P to maintain the device 100.

The device 100 can also be divided into one or more elements, able to be worn on different parts of the body of the person P, for example on the head, on the wrist or on the torso.

The device 100 comprises acquisition means or elements 130 for acquisition of at least one measurement signal and at least one memory 160. The device 100 may also comprise analysis means 150 or elements for analyzing the measurement signal. The device 100 may finally comprise transmission means or elements 140 designed to emit an acoustic signal which is audible by the person P as will be described later.

The device is for example adapted to be worn by the person P during a working period that may extend over a period of several minutes to several hours, for example at least eight hours.

By “working period” is meant a period during which the measuring device is active and implements a predefined work operation, for example an acquisition of a measurement signal S which representative of a physiological signal of the person P. The person P can, for its part, be inactive, for example asleep during the working period. The measuring device can further implement other operations, for example analysis or data transmission, out of the working period.

The working period may for example correspond to a sleeping period of the person P, especially when the measuring device is a sleep monitoring and/or stimulation device.

The device 100 may further comprise a battery 180. The battery 180 may in particular be able to feed the acquisition means 130, the transmission means 140 and the analysis means 150, the memory 160 and the communication module.

The battery 180 is for example able to provide energy without being recharged throughout the working period, for example over a period of several hours without having to be recharged, for example at least eight hours.

The device 100 can in particular operate in an autonomous manner during the working period.

By “autonomous” is meant that the device can operate during the working period, and in particular implement brain waves acquisition and/or stimulation operations as described below, without communicating with the processing server 200, in particular without communicating with the processing server 200. In particular, it is meant that the device can operate during the working period without the need to be recharged with electrical energy and without the need to be structurally connected to an external device such as a fastener or a power supply.

In this way the device 100 is adapted to be used in the daily life of a person P without imposing undue burden.

To enable the brain waves acquisition and/or stimulation operations implementation, the acquisition means 130, the transmission means 140, the analysis means 150 and the memory 160 are moreover functionally connected between them and able to exchange information and instructions.

For this purpose, the acquisition means 130, the transmission means 140, the analysis means 150 and the memory 160 are mounted on the support element 120 so as to be close to one another so that the communication between these elements 130, 140, 150, 160 is especially fast and at a high throughput. The battery 180 can also be mounted on the support member 120.

The memory 160 may be permanently mounted on the support member 120 or may be a removable module, for example a memory card such as an SD card (acronym for the term “Secure Digital”).

The memory 160 is able to record operating data of the device 100. Said operating data will be detailed in the following description and may comprise at least one of the following elements: raw measurement data comprising a measurement signal S as acquired by the means 130, processed measurement data determined from the measurement signal S.

The memory 160 is able to be dynamically updated while the device 100 is being operated.

The working step is illustrated in FIG. 6 and can thus firstly comprise an at least one measurement signal S acquisition sub-step by means of acquisition means 130.

The measurement signal S can in particular be representative of a physiological electrical signal E of the person P.

The physiological electrical signal E may for example comprise an electroencephalogram (EEG), an electromyogram (EMG), an electrooculogram (EGG), an electrocardiogram (ECG) or any other measurable biosignal on the person P.

For this purpose, the acquisition means 130 comprise for example a plurality of electrodes 130 adapted to be in contact with the person P, and in particular with the skin of the person P to acquire at least one measurement signal S representative of a physiological electrical signal E of the person P.

The physiological electrical signal E advantageously comprises an electroencephalogram (EEG) of the person P.

To this end, in one embodiment of the invention, the device 100 comprises at least two electrodes 130 including at least one reference electrode 130a and at least one EEG measuring electrode 130b.

The device 100 may further comprise a ground electrode 130c.

In a particular embodiment, the device 100 comprises at least three EEG measurement electrodes 130c, so as to acquire physiological electrical signals E comprising at least three electroencephalogram measuring channels.

The EEG measurement electrodes 130c are for example disposed on the surface of the scalp of the person P.

In other embodiments, the device 100 may further comprise an electrode for measuring the EMG and, optionally, an EOG measuring electrode.

The measurement electrodes 130 may be reusable electrodes or disposable electrodes. Advantageously, the measurement electrodes 130 are reusable electrodes so as to simplify the daily use of the device.

The measurement electrodes 130 may be, in particular, dry electrodes or electrodes covered with a contact gel. The electrodes 130 may also be textile or silicone electrodes.

The acquisition means 130 may also include acquisition devices for the acquisition of measuring signals S which are not only electrical.

A measurement signal S can thus be, in general, representative of a physiological signal of the person P.

The measurement signal S may in particular be representative of a non-electrical or non-completely electrical physiological signal of the person P, for example a cardiac work signal, such as a heart rate, a body temperature of the person P or movements of the person P.

To this end, the acquisition means 130 may comprise a heart rate detector, a body thermometer, an accelerometer, a breathing sensor, a bioimpedance sensor or a microphone.

The acquisition means 130 may also include measurement signal acquisition devices S representative of the person P environment.

The measurement signal S can thus be representative of a quality of the air surrounding the person P, for example a carbon dioxide or oxygen level, or a temperature or ambient noise level.

Finally, the acquisition means 130 may include user input devices allowing the person P to enter information. For example the user can indicate a subjective index of night quality. The measurement signal S can then be representative of information provided by the person P.

The measurement signal S thus obtained can thus constitute raw measurement data in the sense of the present description.

Moreover, in an embodiment of the invention, the measurement signal S acquisition sub-step also comprises a preprocessing of the measurement signal S.

The preprocessing of the measurement signal S may for example comprise at least one of the following preprocessings:

a frequency filter, for example a frequency and/or wavelet filtering of the measurement signal S in a temporal) frequency range of interest, for example a frequency range comprised in a span from 0.3 Hz to 100 Hz,

a frequency and/or wavelet filtering of parasitic frequencies of the measurement signal S, for example able to filter at least at least one parasitic frequency of the measurement signal S, for example a parasitic frequency belonging to a frequency range from 0.3 Hz at 100 Hz,

an elimination of predefined artifacts of the measurement signal S.

The preprocessing of the measurement signal S may also include preprocessings such as:

an amplification, for example an amplification of the measurement signal S by a factor ranging from 10̂3 to 10̂6, and/or

a sampling of the measurement signal S by means of an analog-digital converter able, for example, to sample the measurement signal S with a sampling rate of a few hundred Hertz, for example 256 Hz or 512 Hz.

Such preprocessing of the measurement signal S may for example be implemented by an analog module or a digital module belonging to the acquisition means 130. Thus, in particular, the acquisition means 130 may comprise active electrodes capable of carrying out one of the preprocessings detailed above.

The measurement signal S obtained as a result of the preprocessing may also constitute raw measurement data within the meaning of the present description.

The working step of the present method may also include a measurement signal S processing sub-step.

The measurement signal S processing sub-step makes it in particular possible to determine processed measurement data.

To implement this processing sub-step, the device comprises analysis means 150 capable of analyzing the measurement signal S.

The analysis means 150 may, for example, implement one or more predefined pattern recognition algorithms in the measurement signal S, for example slow wave patterns, sleep spindle patterns, K-complex patterns, or patterns associated with the waking and/or with the movements of the person.

The processed measurement data can thus comprise indicators relating to said predefined patterns, including a predefined pattern start time, duration, frequency and/or amplitude, and/or a number or frequency of a pattern which is predefined during the working period.

The processed measurement data can also comprise other synthetic data determined from the measurement signal S, for example average values of the signal, spectral means or other digital indicators that can be determined from the measurement signal S.

The processed measurement data may also include higher level indicators such as sleep phases or waking or micro-waking times.

The processed measurement data can also comprise the lossy compressed measurement signal, for example a wavelet compression. By “raw measurement signal” is meant the measurement signal S and possibly the measurement signal compressed by a lossless compression algorithm, for example an entropic compression of the zip type.

The processed measurement data are thus determined from the measurement signal S and may in particular not include the raw measurement signal S itself. In this way, the processed measurement data may be smaller than the size of the raw measurement data, for example a size at least ten times smaller than the size of the raw measurement data or at least 100 times smaller, in particular at least ten times smaller than the size of the measurement signal S.

In a first exemplary embodiment, a frequency spectrum of the measurement signal S can be determined. The predefined shapes are then determined from a frequency spectrum energy variation in predefined frequency bands such as for example an alpha (8 12 Hz), beta (>12 Hz), delta (<4 Hz) or theta (4 7 Hz) waves frequency band.

A frequency spectrum energy in one or more of said frequency bands can be calculated, for example using a fast short-term Fourier transform.

In another exemplary embodiment, possibly combinable with the first exemplary embodiment indicated, the predefined shapes can be determined directly in the temporal form of the measurement signal S, in particular by searching for one or more predefined patterns in the measurement signal S.

Thus, for example, slow oscillations and K-complexes can be detected by searching for consecutive zeros spaced less than about one second apart and seeking a maximum peak to peak.

When said peak-to-peak maximum exceeds a certain threshold, a slow wave or K-complex pattern can then be identified.

The analysis means 150 can also analyze a measurement signal S representing a level of muscular work, for example an electrooculogram. In this case, the analysis means 150 can for example calculate a running average of a variation of the eyes movement.

The analysis means 150 can also implement an automatic identification algorithm from the measurement signal S. Such an automatic identification algorithm is for example defined during a preliminary automatic learning step.

By “automatic identification algorithm” is meant an algorithm adapted to identify and automatically classify patterns in measurement data, for example by associating a class with them, based on qualitative or quantitative rules characterizing the measurement data.

Said class associated with the measurement data may be selected from a class database, or may be an interpolated value from a class database.

A “class” can thus be for example an identifier, for example an alphanumeric identifier of a predefined pattern, or a numerical value, or where appropriate an integer or real value.

The class obtained can identify a predefined pattern in the measurement signal S, for example to identify a K-complex pattern or a spindle.

Such an automatic identification algorithm may for example implement a neural network, a support vector machine, a decision tree, a random decision tree forest, a genetic algorithm or further factor analysis, linear regression, Fisher discriminant analysis, logistic regression, or other known methods from the classification field.

Such an algorithm may include a plurality of parameters that define the qualitative or quantitative rules from which the automatic identification algorithm can automatically detect and classify the measurement data. Such parameters are, for example, the weights of certain neurons or of all neurons for an algorithm implementing a neural network. The parameters of the automatic identification algorithm may for example be predefined during a supervised automatic learning step, or more or less automatically determined, for example by the implementation of an automatic learning step which can be semi-supervised, partially supervised, unsupervised or a reinforcement learning step. The class database may also be predefined during such a learning step. Such an automatic learning step can be implemented from a measurement data learning sample.

Finally, the working step can comprise an acoustic signal A transmission sub-step constituting a person P brain waves stimulation operation.

For this purpose, the device 100 may comprise transmission means 140 designed to emit an acoustic signal A, audible by the person, and synchronized with a predefined brain wave temporal pattern M1 of the person if it is estimated that the person is in a state fitting for stimulation.

For this purpose, the transmission means 140 comprise, for example, at least one acoustic transducer 110 and a control electronics 190.

The control electronics 190 is particularly suitable, in soft real-time, to receive the measurement signal S from the acquisition means 130 and to control the transmission by the acoustic transducer 110 of an acoustic signal A synchronized with a temporal pattern predefined T of a slow brain wave of the person P.

By “soft real-time” is meant an implementation of the stimulation operation such as temporal constraints on this operation, in particular on the duration or repetition frequency of this operation, are respected on average over a predefined total implementation period, for example a few hours. In particular, the implementation of said operation may at times exceed said temporal constraints as long as the average operation of the device 100 and the average implementation of the method respects them over the total predefined implementation time. In particular, time limits may be predefined beyond which the implementation of the stimulation operation must be stopped or paused.

To allow such a flexible implementation in soft real-time, a maximum distance between the acquisition means 130, the transmission means 140, the analysis means 150 and the memory 160 may be less than about one meter and preferably less than a few tens of centimeters. In this way, a sufficiently fast communication between the elements of the device 100 can be guaranteed.

The acquisition means 130, the transmission means 140, the analysis means 150 and the memory 160 may for example be housed in the cavities of the support element 120, clipped onto the support element 120 or else fixed to the support element 120 for example by gluing, screwing or any other suitable fastening means. In one embodiment of the invention, the acquisition means 130, the transmission means 140, the analysis means 150 and the memory 160 may be removably mounted on the support member 120.

In an advantageous embodiment of the invention, the control electronics 190 is functionally connected to the acquisition means 130 and to the acoustic transducer 110 via wire links 170. In this way, the exposure of the person P to electromagnetic radiation is reduced.

The acoustic transducer or transducers 110 are able to emit an acoustic signal A stimulating at least one inner ear of the person P.

In a first embodiment, an acoustic transducer 110 is an osteophonic device stimulating the inner ear of the person P by bone conduction.

This osteophonic device 110 may for example be able to be placed close to the ear, for example above as shown in FIG. 1, in particular on a skin area covering a cranial bone.

In a second embodiment, the acoustic transducer 110 is a speaker stimulating the inner ear of the person P through an ear canal leading to said inner ear.

This speaker may be disposed outside the ear of the person P or in the ear canal.

The acoustic signal A is a modulated signal belonging at least partially to a frequency range audible by a person P, for example the range from 20 Hz to 30 kHz.

The control electronics 190 receives the measurement signals S from the acquisition means 130, possibly preprocessed as detailed above.

If the measurement signals S received by the control electronics 190 are not preprocessed, the control electronics 190 may in particular implement one and/or the other of the preprocessings detailed above.

The control electronics 190 is then able to implement a brain wave stimulation operation of the person P, an operation which will now be described in more detail.

Brain waves can in particular be slow brain waves.

By “slow brain wave” is meant in particular an electrical brain wave of the person P having a frequency of less than 5 Hz and greater than 0.3 Hz. By “slow brain wave” can be meant an electrical brain wave of the person P having a peak-to-peak amplitude of, for example, between 10 and 200 microvolts. In addition to the very low frequency waves below 1 Hz, slow brain waves are also understood to mean, in particular, delta waves of higher frequencies (usually between 1.6 and 4 Hz). By “slow brain wave” can also be meant any type of wave having the frequency and amplitude characteristics mentioned above. For example, the phase 120 sleep waves referred to as “K-complexes” can be considered as slow brain waves for the purpose of the invention.

In general, the implementation of the invention may for example take place during a sleep phase of the person P (as identified for example in the AASM standards, acronym for “American Academy of Sleep Medicine”), for example a deep sleep phase of the person P (commonly known as stage 3 or stage 4) or during other phases of sleep, for example during light sleep of the person (usually called stage 2).

The invention can also be implemented during an awakening phase, sleep or awakening of the person P. Brain waves can then differ from slow brain waves.

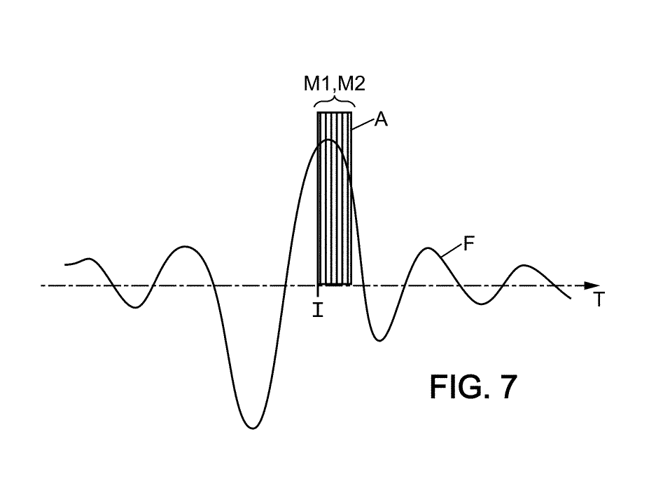

In order to implement the brain wave stimulation operation, the control electronics 190 is, for example, able, from the measurement signal S, to first determine a temporal form F of a slow brain wave C such as that illustrated in FIG. 7.

In a first embodiment, the temporal form F is a series of sampled points of amplitude values of the measurement signal S, possibly preprocessed as mentioned above, said series of measurement points possibly being interpolated or resampled.

In a second embodiment, the temporal form F is a series of amplitude values generated by a phase locked loop (commonly referred to as PLL).

The phase-locked loop is such that the instantaneous phase of the temporal form F at the output of said loop is locked (or slaved) with regard to the instantaneous phase of the measurement signal S.

The phase locked loop can be implemented by analog means or digital means.

It is therefore understood that the temporal form F is a representation of the brain wave C which can be obtained directly or by a phase-locked loop which allows obtaining a cleaner signal. In particular, the instantaneous phase of the temporal form F and of the brain wave C are synchronized temporally. In the present description, therefore, the term “brain wave C” is used to mean the values taken by the temporal form F.

From this temporal form F, the control electronics 190 is able to determine at least a synchronization time instant I between a predefined temporal pattern M1 of slow brain wave C and a predefined temporal pattern M2 of the acoustic signal A.

Then, the control electronics 190 is able to control the acoustic transducer 110 so that the predefined temporal pattern M2 of the acoustic signal A is emitted at the synchronization time instant I.

The predefined temporal pattern M1 of slow brain wave C is therefore an amplitude and/or phase values pattern of the temporal form F which represents the slow brain wave C. In particular, the predefined temporal pattern M1 may be a succession of phase values of the temporal form F and may therefore be in particular independent of the absolute amplitude value of the temporal form F.

The predefined temporal pattern M1 can also be a succession of relative amplitude values of the temporal form F. Said relative values are for example relating to a maximum amplitude of the predefined or stored temporal form F.

In an embodiment of the invention, the predefined temporal pattern M1 can thus for example correspond to a local temporal maximum of the slow brain wave C, a local temporal minimum of the slow brain wave C or a predefined succession of at least one local temporal maximum and at least one local temporal minimum of the slow brain wave C.

The predefined temporal pattern M1 may also correspond to a portion of such a maximum, minimum or of such a succession, for example a rising edge, a falling edge or a plateau.

In the same manner, the predefined temporal pattern M2 of the acoustic signal may be an amplitude and/or phase values pattern of the acoustic signal A.

In a first embodiment, the acoustic signal is for example an intermittent signal as illustrated in FIG. 7. This intermittent signal is for example emitted for a shorter duration than a period of a slow brain wave. The duration of the intermittent signal is for example less than a few seconds, preferably under one second.

In an example given for purely indicative and non-limiting purposes, the acoustic signal A is for example a 1/f -type pink noise pulse with a time duration of 50 to 100 milliseconds with a rise and fall time of a few milliseconds. Still in a non-limiting manner and to make things clear, in this example the predefined temporal pattern M1 of slow brain wave C can for example correspond to a rising edge of a local maximum of the slow brain wave C. The predefined temporal pattern M2 of the acoustic signal A can then be for example a rising edge of the pink noise pulse. In this example, the synchronization time instant I between the predefined temporal pattern M1 of slow brain wave C and the predefined temporal pattern M2 of the acoustic signal A can for example be defined so that the rising edge of the pink noise pulse A and the rising edge of the local maximum of the slow brain wave C are synchronized, that is to say concomitant.

In another embodiment, the acoustic signal A may be a continuous signal. The duration of the acoustic signal A can then in particular be greater than a period of the slow brain wave C. By “continuous signal” is meant in particular a signal of great duration as compared to a period of the slow brain wave C.

In this embodiment, the acoustic signal A can be temporally modulated in amplitude, frequency or phase and the predefined temporal pattern M2 of the acoustic signal A can then be such a temporal modulation.

Alternatively, the continuous acoustic signal A may be temporally unmodulated, for example in a manner that will now be described.

The device 100 may comprise at least two acoustic transducers 110, in particular a first acoustic transducer 110a and a second acoustic transducer 110b as illustrated in FIG. 2. The first acoustic transducer 110a is able to emit an acoustic signal A1 stimulating a right inner ear of the person P. The second acoustic transducer 110b is able to emit an acoustic signal A2 stimulating a left inner ear of the person P.

In particular, the first and second acoustic transducers 110a, 110b can be controlled in such a way that the acoustic signals A1 and A2 are binaural acoustic signals A. For this purpose, the acoustic signals A1 and A2 may for example be continuous signals having different frequencies.

Such acoustic signals A1, A2 are known to generate intermittent pulses in the person's brain P, in particular called binaural beats.

Still in a non-limiting manner and to make things clear, in this example, the predefined temporal pattern M1 of slow brain wave C may, for example, again correspond to a rising edge of a local maximum of the slow brain wave C. The predefined temporal patterns M2 of the acoustic signals A1, A2 may also be ranges of the acoustic signals A1, A2 corresponding temporally to said intermittent pulses generated in the brain of the person P. In this example, the time instant I of synchronization between the predefined temporal pattern M1 of slow brain wave C and the predefined temporal patterns M2 of the acoustic signals A1, A2 may for example be defined so that an intermittent pulse generated in the brain of the person P is synchronized temporally with the rising edge of the local maximum of the slow brain wave C.

FIG. 7 illustrates an example of predefined temporal patterns M1 and M2.

One and/or the other of a sound level, a duration, a spectrum and a temporal pattern M2 of the acoustic signal A can be predefined and recorded in the memory 160 of the device 100.

Said one and/or other of a sound level, a duration, a spectrum and a temporal pattern M2 of the acoustic signal A can form operating data of the device 100.

More specifically, the operating data may comprise one or more stimulation parameters selected from a list comprising an acoustic stimulation pattern start time, duration, amplitude, spectrum and/or reference of the acoustic signal A.

The acoustic signal A can thus be transmitted according to said operating data.

According to the embodiments and according to the selected time pattern M1, various embodiments can be envisaged to determine the synchronization time instant I.

Likewise, one and/or the other of a brain wave phase of the person and a predefined temporal brain wave pattern M1 of the person P can be predefined and stored in the memory 160 of the device 100.

Said one and/or the other of a brain wave phase of the person and a predefined temporal brain wave pattern M1 of the person P can form operating data of the device 100.

The acoustic signal A can thus be emitted so as to be synchronized according to said operating data.

Furthermore, in order to determine the time instant I, the control electronics 190 may for example compare the amplitude values of the measurement signal S, possibly filtered and/or normalized, with an amplitude threshold.

In the example given above for purely non-limiting purposes, the predefined temporal pattern M1 of slow brain wave C corresponds to a rising edge of a local maximum of the slow brain wave C. A temporal instant I then corresponds to a time instant during which the amplitude threshold is overtaken, or at a predefined duration immediately following such an overrun time. The control electronics 190 can thus control the acoustic transducer 140 so that the predefined temporal pattern M2 of the acoustic signal A is synchronized temporally with said time instant I.

It is well understood that the speed of communication between the acquisition means 130, the acoustic transducer 110 and the control electronics 190 makes it possible in particular to ensure reliable synchronization and optimal implementation of the stimulation operation.

In an embodiment in which the temporal form F is a series of amplitude values generated by a phase locked loop, it is possible to determine said time instant I from said phase locked loop, by threshold detection or by predicting future values of temporal form F.

In this embodiment, the temporal form F may in particular be less noisy than the measurement signal S and may allow a facilitated determination of the synchronization time instant I. In this way, it is thus easier to use the phase values of the temporal form F to identify the time instant I.

As illustrated in FIG. 6, once the working step is over, the method according to the invention can then comprise a first connection test step.

This first connection test step can thus be implemented after the working period.

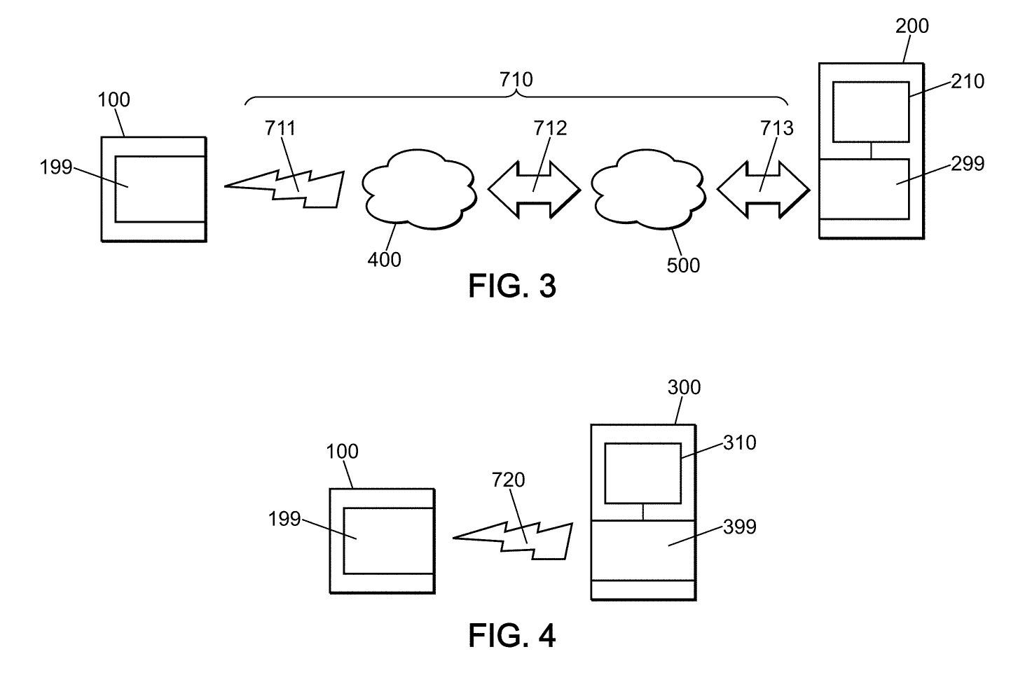

This first connection test step is in particular illustrated in FIG. 3.

During the first connection test step, it is determined whether a primary connection 710 can be established between the measuring device 100 and the data processing server 200.

For this purpose, the measuring device 100 may comprise communication means or elements 199 and the data processing server 200 may also comprise communication means or elements 299.

The communication means 199, 299 of the measuring device 100 and the data processing server 200 may be able to determine whether a primary connection can be established between the measurement device 100 and the data processing server 200, and to transfer data from the measurement device 100 to the data processing server 200, by means of such a primary connection.

The communication means 199 can be mounted on the support element 120 in the manner described above for the acquisition means 130, the transmission means 140 and the analysis means 150. The communication means 199 can be controlled by an electronic device 100, for example the control electronics 190.

The communication means 199 comprise in particular a wireless communication chip.

The communication means 199 may thus comprise a radio frequency communication module, for example a module able to implement a near-field communication, a Bluetooth communication and/or a Wi-Fi communication.

Bluetooth means, in particular, the Bluetooth protocol and the “Bluetooth Low Energy” (BLE) protocol.

The communication means 199 may also include an ultrasonic communication module or an optical communication module, for example embedding a diode.

The communication means 299 of the processing server 200 may for example be means for accessing the Internet, for example wired communication means such as an Ethernet card.

The primary connection 710 may be a wireless connection, at least on the measurement device 100 side.

To this end, the primary connection 710 can be implemented by means of a local wireless network 400 connected to an extended network 500.

The wide area network 500 is for example the Internet.

The local wireless network 400 is for example a corporate wireless network or a home wireless network, in particular a Wi-Fi network connected to the Internet.

The measuring device 100 can thus for example seek to connect to a home wireless network and, from this wireless network, seek to connect to the Internet, and at the same time to the processing server 200 which can also be connected to the Internet.

The primary connection 710 may thus comprise a connection 711 of the measurement device 100 to a local wireless network 400, a connection 712 of the local wireless network 400 to an extended network 500, and a connection 713 of the extended network 500 to the data processing server 200.

The connection 711 between the measuring device 100 and the local wireless network 400 may in particular be a wireless connection.

If a primary connection can be established, a primary transfer step can then be implemented.

This primary transfer step is in particular illustrated in FIG. 3.

The primary transfer step can be implemented by means of said primary connection.

The primary transfer step includes transmitting operating data from the measurement device to the data processing server.

The operating data can be determined from the measurement signal.

The operating data transmitted from the brain wave measuring device to the data processing server during the primary transfer step may in particular comprise raw measurement data as described above, that is to say data comprising the measurement signal S.

If a primary connection can not be established, the method according to the invention may then comprise a second connection test step illustrated in FIG. 4 in particular.

During this second connection test step, it is possible to determine whether a secondary connection 720 can be established between the measuring device 100 and the portable relay device 300.

By “secondary connection” is meant that this secondary connection is implemented if the primary connection described above is not possible to implement, so it is a connection to ensure resilient operation of the system.

The portable relay device 300 is a device transportable by a user and able to communicate with the measuring device and a wireless network.

The portable relay device 300 is for example a base, a mobile phone, a smartphone, an electronic tablet or a laptop.

The portable relay device 300 may in particular comprise communication means or elements 399.

The communication means 399 of the portable relay device 300 may comprise a control chip and a radio-frequency wireless communication module comprising an antenna, an ultrasonic communication module comprising a microphone and/or an optical communication module comprising for example a diode.

For example, a radiofrequency wireless communication module of the communication means 399 may be a module able to implement a near-field communication, a Bluetooth communication and/or a Wi-Fi communication.

If a secondary connection 720 can be established, the method can then include a step of secondary transfer of operating data from the measuring device 100 to the portable relay device 300.

This secondary transfer step is illustrated in FIG. 4.

The secondary connection 720 is a wireless connection between the measurement device 100 and the portable relay device 300. The secondary connection 720 may for example be an ultrasonic connection or a radio frequency connection, such as a Bluetooth connection or a near-field communication.

More specifically, in a particular embodiment of the invention illustrated in particular in FIG. 6, the second connection test step may comprise a first test sub-step during which it is determined whether a radio frequency connection can be established between the measuring device 100 and the portable relay device 300. Such a radio frequency connection may for example be a Bluetooth connection or a near-field communication.

If a radio-frequency connection can be established, the secondary connection is a radio-frequency connection.

If a radio frequency connection can not be established, a second test sub-step can be implemented in the course of which it is determined whether an ultrasonic connection can be established between the brain waves measuring device 100 and the portable relay device 300.

If an ultrasonic connection can be established, the secondary connection is an ultrasonic connection.

In this particular embodiment, the communication means 399 of the portable relay device 300 may comprise both a radio frequency wireless communication module and an ultrasonic communication module. By analogy, the communication means 199 of the measuring device 100 may comprise both a radiofrequency wireless communication module and an ultrasonic communication module.

The secondary connection can thus be a wireless connection.

The secondary transfer step can be implemented by means of said secondary connection.

The secondary transfer step includes transmitting operating data from the measuring device 100 to the portable relay device 300.

The operating data can be determined from the measurement signal.

The operating data transmitted from the measurement device 100 to the portable relay device 300 during the secondary transfer step may comprise processed measurement data as described above. In particular, it is possible that said operating data transmitted from the measuring device 100 to the portable relay device 300 only comprise processed measurement data and not the measurement signal S.

Thus, for example, said operating data transmitted from the measuring device 100 to the portable relay device 300 may have a size at least ten times smaller than a size of the raw measurement data including the measurement signal S.

In this way, it is possible to implement a relatively fast local communication between the measuring device 100 and the portable relay device 300 despite the limited speeds of the local communication protocols such as the Bluetooth, near-field or ultrasonic connections.

If a secondary transfer step has been implemented, the method can then comprise a third connection test step, during which it is determined whether a tertiary connection 730 can be established between the portable relay device 300 and the data processing server 200.

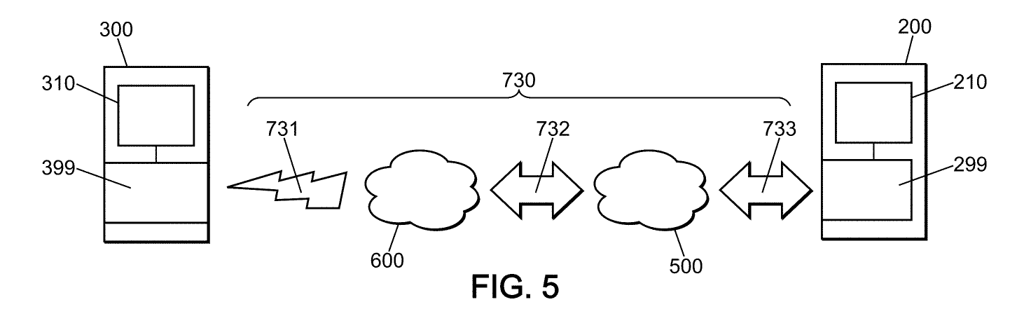

This third connection test step is illustrated in FIG. 5.

By “tertiary connection”, it is meant that this tertiary connection is implemented if the primary connection described above is not possible to implement and if the secondary connection has been implemented. It is therefore a connection to ensure the resilient operation of the system.

The tertiary connection 730 may be a wireless connection, at least from the portable relay device 300.

To this end, the tertiary connection 730 can be implemented by means of a local wireless network 600 connected to an extended network 500.

The wireless network 600 may be a cellular network such as a mobile telephone network.

The wireless network 600 may also be a local wireless network, for example a corporate wireless network or a home wireless network, in particular a Wi-Fi network connected to the Internet.

The tertiary connection 730 may thus comprise a connection 731 of the portable relay device to a wireless network 600, a connection 732 of the wireless network 600 to an extended network 500, and a connection 733 of the extended network 500 to the processing server 200.

The connection 731 between the portable relay device 300 and the wireless network 600 may in particular be a wireless connection.

If a tertiary connection can be established, the method can then include a tertiary transfer step of the operating data of the portable relay device 300 to the data processing server 200, by means of said tertiary connection.

The third connection test step and the tertiary transfer step of the operating data from the portable relay device to the data processing server can be implemented using the communication means 299, 399 of the portable relay device 300 and the data processing server 200.

The operating data transmitted from the portable relay device 300 to the data processing server 200 during the tertiary transfer step may be identical to the operating data transmitted from the measurement device 100 to the portable relay device 300 during the secondary transfer step.

Alternatively, additional data may be added by the portable relay device 300 to the operating data transmitted from the measurement device 100 to the portable relay device 300 during the secondary transfer step to form the operating data transmitted from the portable relay device 300 to the data processing server 200 during the tertiary transfer step.

To this end, the portable relay device may include data processing means or elements 310, including at least one computer chip.

The processing server 200 can thus receive a trace of the working period which has elapsed, even if it does not have the raw operating data.

As can be seen above, the primary connection, the secondary connection and the tertiary connection can all be implemented, at least in part, by wireless communications.

In a particular embodiment of the invention, the portable relay device 300 can be moved between the secondary transfer step and the tertiary transfer step.

The portable relay device 300 may in particular wait to have access to the Internet through a predefined channel to implement the tertiary transfer step.

In particular, the third connection test step may consist in determining whether it is possible to establish, between the portable relay device and the data processing server, a tertiary connection which is a connection through a network local wireless, for example a corporate wireless network or a home wireless network, especially a Wi-Fi network connected to the internet.

If it is only possible to establish, between the portable relay device 300 and the data processing server 200, a tertiary connection which is a connection through a cellular network such as a mobile telephone network, the portable relay device 300 can then choose to wait to transmit the function data to the data processing server 200, so as to limit the costs borne by the user.

In one embodiment of the invention in which the measuring device 100 also implements a brain wave stimulation operation, the operating data transmitted from the brain wave measuring device 100 to the data processing server 200 in the course of the primary transfer step may further comprise at least one stimulation parameter selected from a list comprising an acoustic stimulation pattern start time, duration, amplitude, spectrum and/or reference.

By “a reference of an acoustic stimulation pattern” is meant, for example, an alphanumeric identifier of a predefined stimulation pattern.

In this embodiment, the operating data transmitted from the measurement device 100 to the portable relay device 300 during the secondary transfer step as well as the operating data transmitted from the portable relay device 300 to the data processing server 200 during the tertiary transfer step may also include said at least one stimulation parameter.

In one embodiment of the invention, the data processing server 200 may be able to communicate with a plurality of measurement devices 100 respectively capable of being worn by a plurality of persons P.

The data processing server 200 can thus receive a plurality of operating data respectively associated with the plurality of measuring devices 100.

The data processing server 200 comprises processing means 210, for example one or more calculation chips 210, capable of performing a processing of the operating data, for example able to implement learning algorithms or statistical calculations. The data processing server can thus for example determine statistics or synthetic indices from the operating data.

1-15. (canceled)

16. A method for retrieving operating data from a device for measuring brain waves of a person onto a data processing server, specially adapted for implementation by a system comprising a data processing server, a portable relay device and a device for measuring brain waves of a person, the method comprising at least:
a) a working step in which, during a working period, a measurement signal representative of a physiological signal of the person is acquired by means of the measuring device, and said measurement signal is stored in a memory of said measuring device,
b1) a first connection test step, implemented after said working period, during which it is determined whether a primary connection can be established between the measuring device and the data processing server,
c1) if a primary connection can be established, a step of primary transfer of operating data from the measuring device to the data processing server, by means of said primary connection, said operating data being determined from the measurement signal,
b2) if a primary connection can not be established, a second connection test step, during which it is determined whether a secondary connection can be established between the measuring device and the portable relay device,
c2) if a secondary connection can be established, a step of secondary transfer of operating data from the measuring device to the portable relay device, by means of said secondary connection, said operating data being determined from the measurement signal,
b3) if a secondary transfer step has been implemented, a third connection test step, during which it is determined whether a tertiary connection can be established between the portable relay device and the data processing server,
c3) if a tertiary connection can be established, a tertiary transfer step of the operating data from the portable relay device to the data processing server, by means of said tertiary connection.

17. The method according to claim 16, wherein the portable relay device is a device transportable by a user, in particular a base, a mobile phone, a smartphone, a tablet or a laptop.

18. The method according to claim 16, wherein the primary connection, the secondary connection and the tertiary connection each comprise wireless communication.

19. A method according to claim 16, wherein the primary connection is implemented by means of a local wireless network connected to a wide area network, including a corporate wireless network or a home wireless network connected to the Internet.

20. The method according to claim 16, wherein the secondary connection is a wireless connection between the brain wave measuring device and the portable relay device, including a ultrasonic connection or radio frequency connection such as a Bluetooth connection or near field communication.

21. The method according to claim 16, wherein the tertiary connection is implemented at least in part by means of a wireless network such as a cellular network or a local wireless network connected to the Internet, including a corporate wireless network connected to the Internet or a home wireless network connected to the Internet.

22. The method according to claim 16, wherein the portable relay device is moved between the secondary transfer step and the tertiary transfer step.

23. The method according to claim 16, wherein the second connection test step comprises a first test sub-step in which it is determined whether a radio frequency connection can be established between the brain waves measurement device and the portable relay device,
if a radio-frequency connection can be established, the secondary connection is a radio-frequency connection,
if a radio frequency connection cannot be established, a second test sub-step in which it is determined whether an ultrasonic connection can be established between the brain waves measuring device and the portable relay device,
if an ultrasonic connection can be established, the secondary connection is an ultrasonic connection.

24. The method according to claim 16, wherein the operating data transmitted from the brain wave measuring device to the data processing server during the primary transfer step comprises raw measurement data including the measurement signal.

25. The method according to claim 16, wherein the operating data transmitted during the secondary transfer step and the tertiary transfer step comprise processed measurement data, preferably do not include the measurement signal, even more preferably in which said operating data have a size at least ten times smaller than a size of the raw measurement data including the measurement signal.

26. The method according to claim 25, wherein the processed measurement data is determined by implementing a predefined patterns recognition algorithm for recognition of predefined patterns in the measurement signal, including slow wave patterns, sleep spindle patterns, patterns associated with the waking and/or with the movements of the person,
and wherein said processed measurement data comprises indicators relating to said predefined patterns, including a predefined pattern start time, duration, frequency and/or amplitude, and/or a number or frequency of a pattern which is predefined during the working period.

27. Process according to claim 16, wherein during the working step, an acoustic signal is transmitted, audible by the person, and synchronized with a predefined temporal brain wave pattern of the person,
and the operating data transmitted during the primary transfer step comprises at least one stimulation parameter selected from a list comprising an acoustic stimulation pattern start time, duration, amplitude, spectrum and/or reference,
preferably the operating data transmitted during the secondary transfer step and during the tertiary transfer step also comprise said at least one stimulation parameter.

28. A system comprising a data processing server, a portable relay device and a device for measuring the brain waves of a person,
wherein the measuring device comprises
acquisition elements capable, during a working period, of acquiring at least one measurement signal which is representative of a physiological signal of the person,
a memory capable of storing said measurement signal, and
communication elements suitable for
determining whether a primary connection can be established between the measuring device and the data processing server,
transferring data from the measurement device to the data processing server by means of a primary connection,
determining whether a secondary connection can be established between the measuring device and the portable relay device, and
transferring data from the measuring device to the portable relay device by means of a secondary connection,
wherein the portable relay device comprises communication elements suitable for
determining whether a tertiary connection can be established between the portable relay device and the data processing server,
transferring data from the portable relay device to the data processing server by means of a tertiary connection.

29. Device for measuring the brain waves of a person specifically intended to be integrated in a system according to claim 28, the device comprising
acquisition elements capable, during a working period, of acquiring at least one measurement signal which is representative of a physiological signal of the person,
a memory capable of storing said measurement signal, and
communication elements suitable for
determining whether a primary connection can be established between the brain wave measuring device and a data processing server of a system according to claim 28,
transferring data from the measurement device to the data processing server by means of a primary connection,
determining whether a secondary connection can be established between the brainwave measuring device and a portable relay device of a system according to claim 28, and
transferring data from the measuring device to the portable relay device by means of a secondary connection.

30. The device of claim 29, further comprising transmitting elements adapted to transmit an acoustic signal, audible to the person, and synchronized with a predefined brain wave temporal pattern of the person.
